(19) United States
(12) Patent Application Publication
(10) Pub. No.: US 2016/0089139 A1
(43) Pub. Date: Mar. 31, 2016

(54) Suture Needle Capture Devices and Methods

(71) Applicant: Wake Forest University Health Sciences, Winston-Salem, NC (US)

(72) Inventors: L. Andrew Koman, Winston-Salem, NC (US); Thomas L. Smith, Winston-Salem, NC (US)

(21) Appl. No.: 14/869,303

(22) Filed: Sep. 29, 2015

Related U.S. Application Data
(60) Provisional application No. 62/056,850, filed on Sep. 29, 2014.

(51) Int. Cl. A61B 17/06

(52) U.S. Cl. CPC A61B 17/06061; A61B 17/06114; A61B 2017/06142; A61B 2017/06052; A61B 17/3494

(57) ABSTRACT

Provided herein are protective devices that can fully capture and enclose a needle for a medical procedure, e.g., a swedged suture needle, such that the needle may safely remain attached to a wound or surgical site via suture material but also prevent accidental needle-stick injuries to medical personnel and patients. Such devices may be employed for temporary or longer periods of time while other phases related to the medical procedure transpire.

### RELATED APPLICATIONS

The present application claims priority to U.S. Provisional Patent Application No. 62/056,850, filed Sep. 29, 2014. The disclosure of U.S. Provisional Patent Application No. 62/056,850 is hereby incorporated by reference in its entirety herein.

### FIELD

The present invention relates to medical devices and prevention of suture needle injuries to medical personnel or patients.

### BACKGROUND

Suture needles are a primary source of accidental stick injuries in the operating room. OSHA, the American College of Surgeons, and many other medical professional organizations have cited risk to patients and medical personnel from accidental needle stick injuries during surgery and trauma repair. Needle sticks have potential to expose the patient or medical personnel to wound bacteria and other contaminants as well as bloodborne pathogens such as bacteria, fungi and viruses.

Percutaneous injuries caused by suture needles are associated with transmission of bloodborne pathogens such as hepatitis B virus (HBV), hepatitis C virus (HCV), and human immunodeficiency virus (HIV) and may cause bacterial infection or injury. Suture needles cause more injuries in the operating room than any other device. Surgeons, nurses, and other healthcare personnel sustain the vast majority of these injuries in the operating room. While thousands of these injuries are reported each year, the true incidence and prevalence is unknown because many incidents are unreported.

In some surgical cases, it is medically desirable for sutures to be placed in one side of ligaments, fascia, tendons, capsule, bone, and other structures for future completion of the repair or reconstructive process. Placing the suture with the needle left attached for future completion of the repair can allow for better definition of the structures, facilitates exposure without compromising future repairs, allows other procedures on deeper structures to be done with facility, and is more exact and efficient. This is particularly advantageous if the initial repair exposure is done with a tourniquet; after letting the tourniquet down, blood, swelling, etc. will not interfere with identification of the structures to be repaired. Using a factory attached “swedged” (or “swaged”) needle has significant advantages in terms of needle size, time, and efficiency. Thus, it would be particularly beneficial to be able to temporarily store an original swedged needle still attached to the suture while completing the procedure in a manner such that the needle cannot injure the patient or any medical personnel.

Various circumstances require surgical or trauma wounds to be closed in multiple stages, to facilitate later closure while completing procedures or reconstructions beneath the structure to be repaired. In addition, bleeding or swelling may interfere with optimal suture placement prompting suture placement during initial exploration or the approach and the ability to repair later, as in cases where swelling must subside or some healing has begun. A swedged suture needle, which is narrow and has suture material fixedly attached in the factory, is often used for such procedures. When a surgical or trauma site needs to be closed in multiple stages, it is desirable to not tie the suture and to leave the swedged suture needle attached to the unfinished suture so that the repair may be completed at a later time. However, leaving an unprotected needle in the operative field with the risk of accidental needle-stick injuries to doctors, nurses, and patient is inappropriate. Sometimes, hemostats are clamped over the needle tip, or the needle is inserted into styrofoam. However, needles are too easily dislodged from these settings, and they provide incomplete protection.

Alternatively, the swedged suture needles are sometimes cut off by the physician or nurse after a stage of suturing, which later requires the suture material to be manually threaded through one or more additional “eye” needles to continue the repair. The needle is small but provides a real additional cost; the staff must find the correct sized needle (decreased efficiency), there may be additional tissue damage from a larger/broader needle (at the eye); and there is a risk of the needle falling off. Swedged needles are fixedly attached to the suture material in the factory and do not have an eye. Swedged needles used for tendon and ligament repair are usually “atraumatic” and are typically curved. A “free” needle, by contrast, has an “eye” or hole that must be threaded by the user. Eye needles may cause more trauma to the tissue due to added girth around the eye of the needle and drag from the double strand of suture material. Consequently, eye needles are not as well-suited for surgical reconstruction of traumatic injuries as swedged suture needles. Specifically, eye needles present several disadvantages: they are time-consuming to thread, they may become unthreaded during use, and they are thicker at the eye and require a double strand of suture material to be pulled through tissue, both of which contribute to creating a larger hole with additional tissue disruption. Therefore, cutting off a swedged suture needle when suturing is unfinished is not optimal.

A desirable alternative to address this problem is a protective device that may fully capture and enclose a swedged suture needle such that it may remain attached to the surgical site via the suture material, to prevent physicians, nurses or patients from accidental needle-stick injuries while personnel attend to other phases of the surgical procedure or trauma repair, change dressings, allow time for healing, etc. Such a device can reduce needle stick injuries and eliminate the need to cut off swedged needles where suturing is unfinished, allowing a suture needle to be safely left within the operating field for future use.

As such, there is an unmet need for a device to allow suture needles to be safely left in the operating field for subsequent use by surgeons and scrub personnel, while protecting both personnel and patients from accidental injuries.

### SUMMARY

In some aspects, the invention described herein includes a device for storing a needle that is fixedly attached at one end to a suture material, comprising a container for enclosing the needle and a first portion of the suture material, wherein the container comprises a component for securing the needle inside the container, and a mechanism for opening and closing the container, wherein the container is configured to allow a second portion of the suture material to extend from the container when the container is in the closed position and contains a needle.

In some embodiments of the device, the needle is a surgical suture needle. In some embodiments, the needle is a swaged needle. In some embodiments, the device includes a component inside the container that is configured to be pierced by the end of the needle. In some embodiments, the component for securing the needle and the first portion of the suture material inside the container is configured to contain the needle between two elements that secure the needle in a fixed position. In some embodiments, the component configured to contain the needle between the two securing elements includes at least two protrusions configured to contain the needle between the protrusions.

Some embodiments of the device further include a component for securing the first portion of the suture material in a fixed position in the container. In some embodiments of the device, the mechanism for opening and closing the container includes a component for securing the container in a closed position. In some embodiments, the container is configured to comprise a top portion and a bottom portion connected to the mechanism for opening and closing the container. In some embodiments, the top portion and a bottom portion are configured as mirror images of each other.

In some embodiments of the device, the mechanism for opening and closing the container includes a hinge. In some embodiments, the mechanism for opening and closing the container includes at least one handle. In some embodiments, a first handle is attached to a first portion of the container and the second handle is attached to a second portion of the container.

In some embodiments of the device, the first portion of the device and the second portion of the device are connected by a cylindrical component configured to allow inward facing surfaces of the first and second portions of the container to be positioned adjacent to one another so as to close the container.

In some aspects of the device, at least part of the container is sterile. Some aspects further include a latch or lock. In some embodiments of the device, pressure must be applied to the device to open it. In others, pressure must be applied to the device to close it.

Some embodiments further include a notch where the middle part of the needle is exposed when the needle is captured in the closed device.

Some aspects of the invention described herein include methods for temporarily storing a suture needle attached to suture material within a device as described herein, including steps of inserting a suture needle and some portion of attached suture material into the capture device and closing the capture device. In some methods, at least the sharp point of the needle is secured within the device. In some embodiments, at least a part of the suture material is secured within the device. In some methods, the needle is fully enclosed within the device.

### BRIEF DESCRIPTION OF FIGURES

The present invention may be better understood by referring to the following non-limiting figures.

FIG. 1 shows a clamshell type capture device according to one embodiment of the invention.

FIG. 2 shows the clamshell capture device of FIG. 1 in fully open (left panel) and closed (right panel) views.

FIG. 3 shows three different views of a rectangular capture device with rubber bumpers extending from both halves and rubber fins extending from the bottom half near the hinge, according to one embodiment of the invention.

FIG. 4 shows a reverse clamp capture device resembling the mouth of an animal (“duck bill”), according to one embodiment of the invention.

FIG. 5 shows the capture device of FIG. 4 in open (left panel) and closed (right panel) positions.

FIG. 6 shows a capture device wherein the middle of the needle is exposed when the device is closed, according to one embodiment of the invention.

FIG. 7 shows a photograph of a surgeon placing a suture needle in the device of FIG. 6.

FIG. 8 shows a “twist top” capture device according to one embodiment of the invention.

FIGS. 9A and 9B show closed and open views of a prototype with scissor-type locking forceps handles, according to one embodiment of the invention.

FIGS. 10A and 10B show open and closed views of a prototype similar to that in FIG. 9, with scissor-type locking forceps handles, but having a flattened container for storage needs, according to one embodiment of the invention.

FIG. 11 shows close up detail of an end stop mechanism for the prototypes shown in FIGS. 9 and 10, according to embodiments of the invention.

FIG. 12 shows a locking mechanism used with a scissor-type locking forceps handle, for the prototypes shown in FIGS. 9 and 10, according to embodiments of the invention.

### DETAILED DESCRIPTION

The methods, systems, and devices of the present invention may be advantageously utilized in medical treatment protocols for humans and/or in veterinary applications.

The following description recites various aspects and embodiments of the present invention. No particular embodiment is intended to define the scope of the invention. Rather, the embodiments merely provide non-limiting examples of various methods, devices, and systems that are at least included within the scope of the invention. The description is to be read from the perspective of one of ordinary skill in the art; therefore, information well-known to the skilled artisan is not necessarily included.

### DEFINITIONS

Where the definition of terms departs from the commonly used meaning of the term, applicant intends to utilize the definitions provided below, unless specifically indicated.

For purposes of the present invention, it should be noted that the singular forms, “a,” “an” and “the,” include reference to the plural unless the context as herein presented clearly indicates otherwise.

“Suture needle,” as used herein, refers to a medical instrument used to close an incision or wound with suture material (e.g., to make stitches).

A “swedged” (or “swaged”) needle, as used herein, refers to a needle having suture material attached in the factory, such that the needle has no eye.

“Suture material,” as used herein, refers to any of numerous different thread-like materials used for stitching in medical procedures, such as for stitches to close incisions or wounds. Examples include plain catgut, chromic catgut, polyglycolide, polydioxanone, and others.

“Needle driver” or “needle holder,” as used herein, refers to a surgical or medical procedure instrument used to hold and guide a needle, such as a suture needle, during surgical or other medical procedures. Any of the wide variety of needle drivers may be used as the needle driver or holder as described herein.

“Handles,” as used herein, refers to any extension designed for gripping and/or manipulating a medical device using human hands.

“Scissor-type locking forceps handles,” as used herein, refers to the common handle design found in a wide variety of medical instruments, having handles that resemble scissor handles.

“Fixedly attached,” as used herein, refers to the attachment aspect of factory-prepared swedged suture needles. Swedged needles have no eye, and the suture material strongly adheres to the end of the needle without the looping of thread as through traditional sewing needles.

### Devices

Provided herein are protective devices that can fully capture and enclose a swedged suture needle, such that it may safely remain attached to a wound or surgical site via the suture material but also prevent accidental needle-stick injuries to medical personnel and patients. Such devices may be employed for temporary or longer periods of time while other phases of the medical procedure transpire. In some circumstances the temporary storage of the needle may be for one or more minutes, as when other medical implements are being retrieved or medical personnel are changing positions or transferring duties to other medical personnel. In other circumstances, the needle may be stored in a capture device for longer periods of time, e.g., to allow time for healing.

Thus, embodiments of the invention comprise a suture needle capture device for temporarily storing a suture needle for future completion of the repair or reconstruction, comprised of an opening for inserting the needle into the device and a mechanism for firmly and safely securing the needle in the device, with the suture still attached to the needle and the wound/patient. Placing the suture with the needle left attached for future completion of the repair allows better definition of the structures, facilitates exposure without compromising future repairs, allows other procedures involving deeper structures to be done with facility, and is more exact and efficient. This is particularly advantageous in the common scenario where the initial repair exposure is distal to a tourniquet, for example. After the tourniquet is let down (i.e., removed), blood, swelling, etc. will not interfere as much with identification of the structures to be repaired.

Using a suture needle capture device, a healthcare provider, such as a surgeon, may set aside a suture needle safely for continued later use, without concern that the patient or any healthcare personnel may inadvertently be injured by the needle. The suture may remain attached to the needle and to the surgical site on the patient or other instrumentation as needed. In some settings, the capture device may be conveniently stored within the bandages of a particular wound for easy retrieval and reuse. Thus in some embodiments, a capture device is flattened when closed for ease of storage within bandages or in another unobtrusive manner.

Thus, in some aspects the invention comprises a suture needle capture device for temporarily storing a suture needle attached to a suture when a surgical wound cannot or will not immediately be fully closed. In some embodiments, the device includes an opening for inserting the needle into the device and a mechanism for firmly securing the needle in the device. While the suture needle is stored in the capture device, the suture material may remain attached to the needle at one end and the surgical site at the other end, extending from the capture device to the wound on the patient. A suture needle capture device may be various sizes and shapes to accommodate all needle sizes and capture preferences. Capture devices according to various embodiments include a range of small containers with hinges that allow a suture needle to be enclosed such that the sharp point is not exposed. A capture device may be sterile so as to maintain sterility of the suture needle and suture material while they are stored. At least the inside of the container part can be sterile.

In some embodiments, the capture device of the invention comprises a hinge. In some embodiments, the hinge is a living hinge. In some embodiments, the hinge is a scissor hinge. In some embodiments, the device comprises a latch. In some embodiments, the latch may also comprise a hinge. Hinges and latches of various designs may be incorporated into different embodiments of the invention.

In some embodiments, the capture device may include symmetrical aspects to facilitate ease of use by either left-handed or right-handed users. In some embodiments of a capture device, two identical or nearly identical halves are hinged together, such that either half may effectively serve as the ‘base’ or the ‘lid.’ In some embodiments, a capture device may be held and manipulated with a single hand or hemostat.

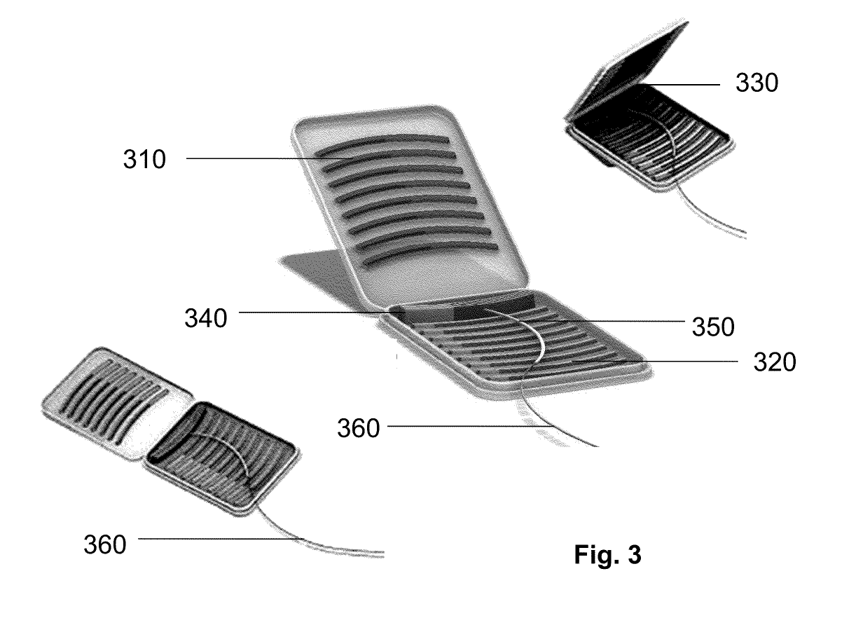

In some embodiments, the invention comprises textured surfaces. For example, features made of a resilient material may be pierced by a needle and/or may grip the needle from one or both sides. In some embodiments, rubber bumpers (shorter and broader segments), fins (taller and thinner segments), or ribs (shorter and thinner segments) may be overmolded onto the inner surfaces of the capture device. In some embodiments, texture features may be incorporated into flat surfaces that contact the inserted needle and/or a part of the suture material to hold them in place. In some embodiments, the stored needle is held firmly between an upper textured surface and a lower textured surface (e.g., as in FIG. 3). For example, resting the needle on ribs raises the needle from the inside surface, which makes it easy to grasp the needle with the needle driver (any tool for holding and manipulating the needle, similar to a hemostat) when being removed from the case. In some embodiments, the texture of the upper surface is designed to match gaps in the texture of the lower surface. For example, upper rubber bumpers may be aligned to meet with lower rubber bumpers, or the bumpers may be staggered in some embodiments for a stronger grip (i.e., for larger, thicker needles).

In some embodiments, the invention comprises a small section of foam, rubber, or other suitable substance for embedding the sharp end of a needle. For example, as the needle is inserted into the capture device, the sharp point may be inserted into a foam or rubber section before the device is closed. In some embodiments, a foam section (or other substance) may be brightly colored to contrast with the rest of the device to facilitate targeting of the sharp point into the foam.

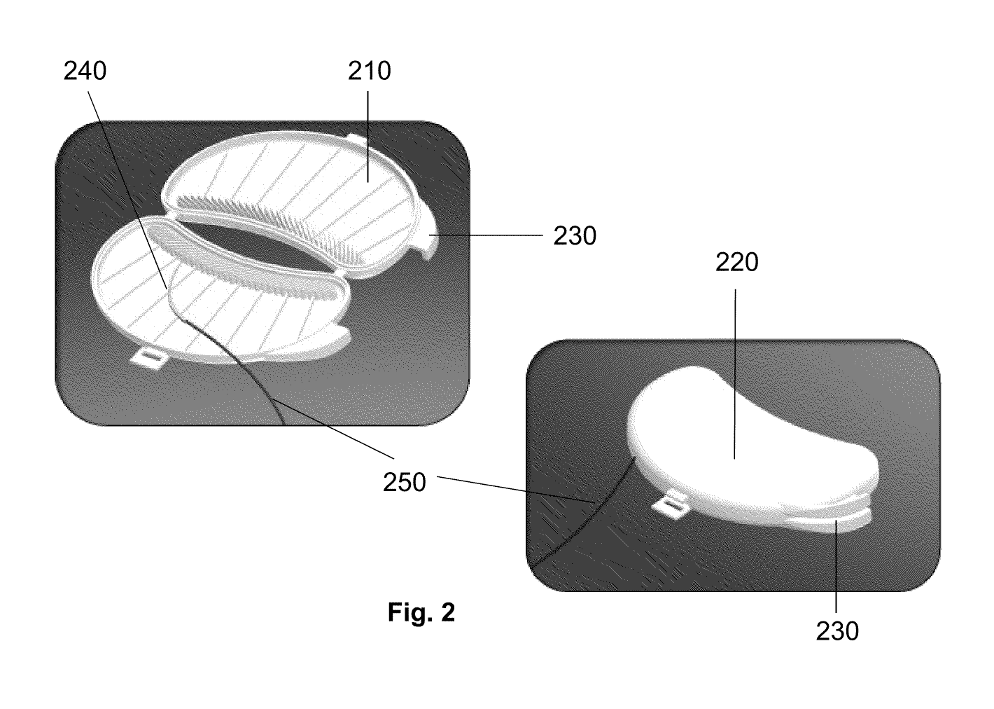

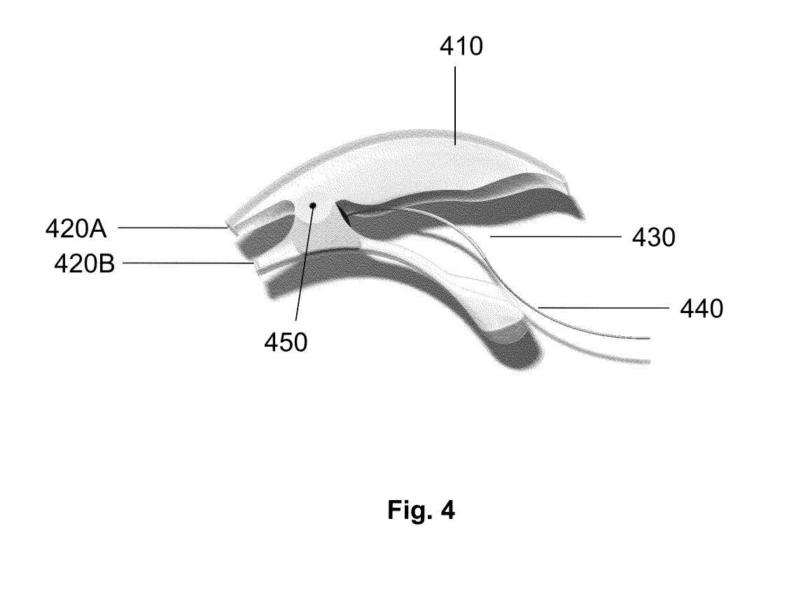

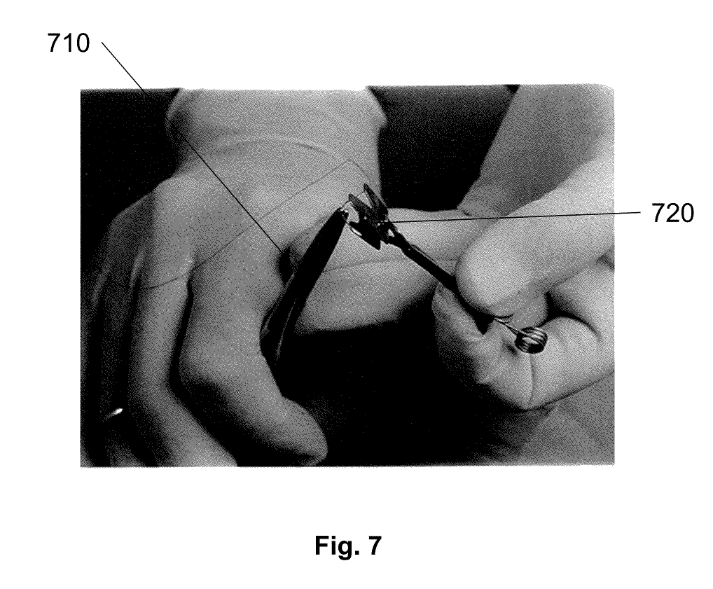

In some embodiments, the default resting position of the capture device is closed. For example, in FIGS. 4 and 7, a user may apply pressure to designated points (e.g., pinch together) in order to open the device and insert a needle. In other embodiments, the capture device may be designed to rest in either the open or closed position (e.g. FIG. 2).

In some embodiments, the capture device resembles the mouth of an animal, such as a duck bill (see FIG. 4). In some embodiments such as the duck bill design and other similar designs, the operational ends may be attached to a hemostat such that the hemostat may be used to pinch the designated points together rather than using fingers (e.g., finger and thumb).

Ideally, all manipulations of a capture device may be accomplished with gloved hands (e.g., surgeon's gloved hand) or a hemostat. In some embodiments, the capture device may be picked up or closed with a single hemostat or hand. In some embodiments, a capture device may include an additional tab, notch, or other feature to facilitate grasping or picking up with a hemostat (see, e.g., FIG. 2). Thus in some embodiments, the capture device may be picked up and closed around the exposed needle together with the needle driver end holding the needle. In other embodiments the device may be picked up and closed with a single gloved hand.

In some embodiments, the capture device may be disposable. Disposable capture devices may be made from a variety of materials, including but not limited to plastic, rubber, foam, polyethylene, polypropylene, and polyurethane. For example, disposable devices could be made from medical grade polyethylene, polypropylene or polyurethane for injection molding conforming to ASTM Volume 13.01 Medical and Surgical Materials and Devices. Some embodiments may include hard or soft plastics, rubber, foam, or some combination of materials. In some embodiments, different materials may be used for different parts or aspects of the device. For example, the container portion of a device may have an outer shell that is harder material so as to prevent any needle from piercing through the container from the inside.

In other embodiments, the capture device may be reusable and therefore more environmentally-conscious. In some reusable embodiments, the capture device may be constructed of durable materials. For example, reusable devices may be made from SAE 304 Stainless Steel conforming to AISI-304-SUS304-1.4301-S30400-Stainless-Steel. Reusable versions may be cleaned and sterilized at the hospital with other reusable instruments.

Some embodiments may include both disposable and reusable components. For example, stainless steel scissor-type locking forceps handles may be reusable (e.g., autoclavable) but also configured to accommodate disposable inserts, e.g., components that include resilient ribs that may be pierced with a suture needle and hold it firmly inside the device.

Thus the suture needle capture device may protect patients, doctors, nurses and other medical personnel from potential accidental needle sticks, allow safe and sterile storage of the needle(s) within the surgical field, and allow for easy retrieval and use when ready to finish a particular suturing task. As such, the suture needle capture device may assist medical facilities in compliance with OSHA's Bloodborne Pathogens standard, published at 29 CFR Section 1910.1030, which protects employees against occupational exposure to bloodborne pathogens.

### Methods

Methods of using such devices are also disclosed. In some embodiments, methods of using capture devices allow medical personnel to temporarily leave the suture needle attached to suture material, which suture material remains attached to the patient's wound or surgical site. In some embodiments a patient's wound or surgical site is in need of further suturing, but the suturing cannot or will not be completed right away. In some embodiments, suturing will be completed after a short time during which the needle may be stored in a suture needle capture device. In other embodiments, significant time may pass before the suturing is completed (e.g., enough time for healing to progress).

Thus in some embodiments, a capture device is employed when a suture needle and the attached suture material need to be set aside for some period of time. In some embodiments, the period of time for storage of the suture needle in the capture device is less than a minute. In some embodiments, the period of time may be 1-15 minutes or 15-60 minutes. In some embodiments the needle may be stored in the capture device for an hour or more, and in some embodiments (for any variety of reasons) days or weeks may pass before the suturing is completed. For example, where an ICU patient is unconscious and/or heavily sedated, such a capture device is less likely to cause discomfort or inconvenience to the patient. In some embodiments a capture device is easily noticed and located when sponges are being used in and around the wound, and when wound dressings need changing.

In some embodiments, placing the needle in the case requires the physician to pierce the needle through some material within the capture device, such as foam, soft plastic, or other resilient material, or to embed the needle at any location along an array of ribs. Once the tip and/or some length of the needle is secured, the needle driver can be released and the capture device may be closed to secure the needle and a portion of the suture material. For example, a clamshell type case capture device can then be pinched shut. The capture device may then be secured, e.g., with a releasable snap latch. This latch can be opened or reopened when the needle is needed again.

A capture device enclosing a suture needle and some part of the attached suture material may be stored near the wound or surgical site to which it is attached (e.g., within bandages covering the wound or surgical site). In some circumstances, the patient will remain in the operating room until the needle is removed from the capture device and suturing is resumed and completed.

In some circumstances, where a patient is transferred out of an operating room along with a capture device storing a needle and suture material, additional measures may be required to maintain the sterility of the suture material extending to the wound or surgical site. For example a capture device and associated needle and suture material may be wrapped into bandages that protect the wound or surgical site. In some circumstances, it may be desirable to enclose most of the excess extending suture material inside a capture device with the suture needle to maintain sterility.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof.

The embodiments illustrated by the figures are described in more detail below.

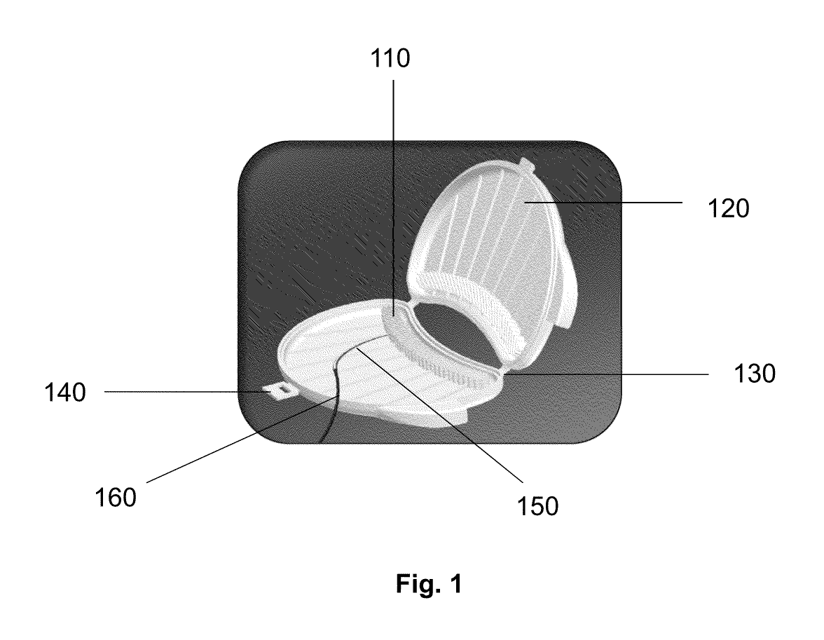

FIG. 1 illustrates a capture device according to one embodiment of the invention. An array of resilient thin plastic ribs 110 within a clamshell type container case 120 are designed to anchor the needle 150 in place when the two halves of the device are pressed together and closed. The user may pierce any of the ribs 110 with the sharp point of the needle 150, and then the needle driver may be released and the case may be pinched closed, with the suture material 160 extending out of the case to the patient/wound (not shown). In some embodiments, the case has a living hinge 130 and a releasable snap latch 140. The latch may also comprise a hinge, and it may be reopened to retrieve the needle when needed. This embodiment may capture the needle completely by enclosing and stabilizing the needle with support ribs. It may also secure a portion of the suture material.

A clamshell type device may be made from polyethylene, polypropylene, or other resilient material such as soft plastics or other suitable materials. In some embodiments a clamshell type device may include some combination of materials. In some embodiments a clamshell type device may also include harder plastic. For example, the halves of the device may include hard plastic on the outermost portions of the device to prevent a needle from piercing through to the outside of the device. The device may be disposable, and it may be made in a range of sizes according to the appropriate suture needle sizes.

FIG. 2 illustrates the capture device of FIG. 1 in an open-flat configuration 210 with suture needle 240 anchored inside, and also in a closed configuration 220, with the suture material 250 extending from the closed case and the latch unfastened. Both views show an extension tab 230 designed to facilitate grasping the device and/or picking it up with a hemostat, for example.

FIG. 3 illustrates a capture device according to another embodiment of the invention. This capture device features over-molded rubber bumpers 310 on the top and bottom 320 flat surfaces of a rectangular or square case. The rubber bumpers of the top half 310 may be aligned with the rubber bumpers on the bottom half 320, such that they meet directly. The rubber bumpers help to hold the needle 350 firmly in place when the case is closed. A hinge 330 connects the bottom and top halves of the case, and over-molded rubber fins 340 located near the hinge 330 are designed to anchor the point of the needle 350 when a user (e.g., surgeon) pierces the fins with the needle point. The suture material 360 extends from the case to the wound/patient.

FIG. 4 illustrates a capture device according to yet another embodiment of the invention. This capture device features a reverse clamp or “duck bill” profile 410, which is opened by pinching the finger tabs 420A and 420B together. When the finger tabs are pinched, the opening 430 appears and a suture needle (inside the device but not visible) may be inserted. Upon release of the finger tabs 420A and 420B, the jaws close and secure the suture needle inside with the suture material 440 extending from the device to the patient. The device shown in FIG. 4 is in a somewhat intermediate position (partially open). A spring-driven hinge 450 closes the opening 430 and separates the finger tabs 420A and 420B when released (or if pressure is not applied). In some embodiments, the operational ends (i.e., finger tabs) may be attached to a hemostat for opening the device instead of using fingers to pinch them together.

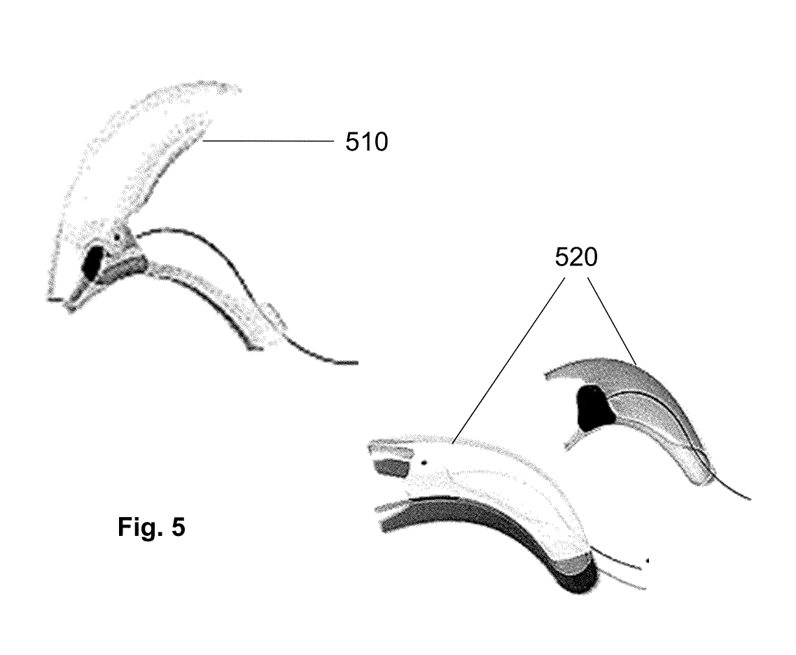

FIG. 5 shows the duck bill device of FIG. 4 in fully open 510 and fully closed 520 positions. All views show suture material extending from the device, indicating that a suture needle has been placed inside (needle not visible in illustrations).

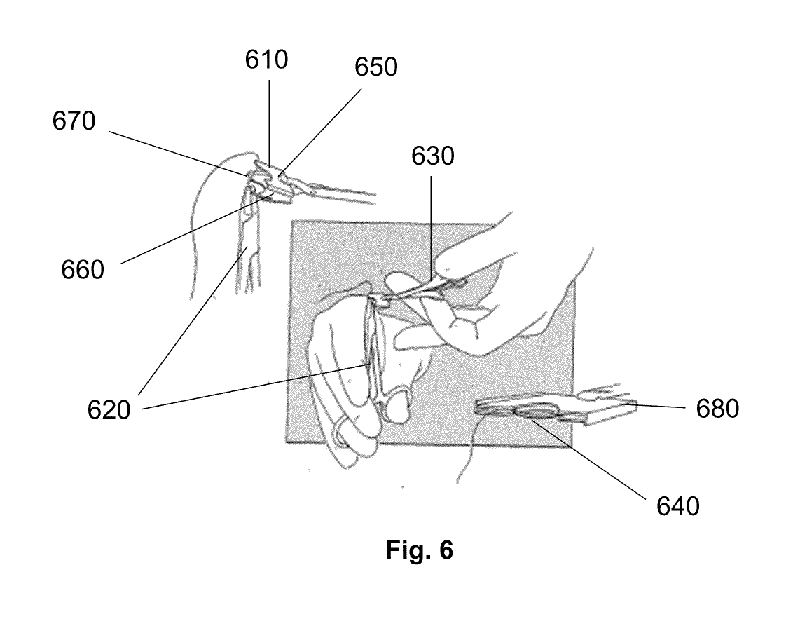

FIG. 6 illustrates a capture device according to yet another embodiment of the invention. This capture device 610 may not completely enclose the suture needle 670, leaving the middle of the needle exposed. For example, the device may comprise two halves 650 and 660 hingedly attached in a square or rectangular flattened shape with a large notch cut out of the opening end to receive the suture needle 670 held by a needle driver 620. The notch accommodates the needle driver grasping the suture needle, even when the capture device is closed 680. As such, the closed capture device 680 leaves a middle part of the needle exposed 640.

To capture the needle, the point of the needle 670 can be placed and grasped between the halves of the device on one side of the notch 610 and the base of the needle can be placed and grasped by the device on the opposite side of the notch. Three views are shown in FIG. 6. The capture device is positioned such that the point of the suture needle is trapped on one side of the device 610, the needle driver 620 (holding the needle) positions the needle 670 in the notch, and the base of the needle is trapped on the other side of the device. Part of the suture material may also be trapped by the device. The middle view shows a surgeon's hands, with one hand holding the suture needle via needle driver 620 and the other hand holding the capture device 630. The capture device may default (rest) in the closed position, such that the user must pinch the handles 630 to open the device (as shown here). Alternatively, the capture device may rest/default in an open position, such that the user pinches the handles to close it. A lock may then be added to secure the device in a closed position. The device is also shown in the closed position 680 with the suture needle 640 trapped inside the device and a suture extending from the device to the wound.

FIG. 7 is a photograph of a user holding the suture needle via needle driver 710 in one hand and the capture device 720 of FIG. 6 in the other hand. The photograph is similar to the middle picture in FIG. 6, except that in the photograph the capture device is in the open position, ready to receive and grasp the suture needle.

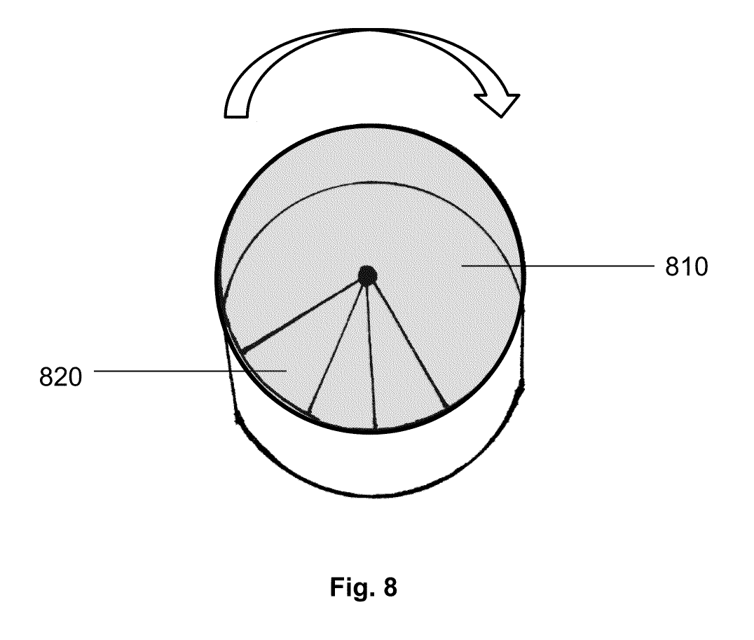

FIG. 8 illustrates a “twist top” capture device according to yet another embodiment of the invention. The top 810 is permanently attached to the shallow cylindrical container, which has another solid sub-top piece just beneath it (not shown for simplicity). The top may be twisted as shown to align an opening in the top piece 810 with an opening 820 in the sub-top piece. The suture needle may be inserted into the opening 820, and then the top may be twisted again to change the alignment such that the opening is covered. The suture material would remain extended from the opening in the top piece to the surgical site (needle and suture material not shown). Optionally a mechanism may ‘lock’ the twistable top piece in place when the device is closed.

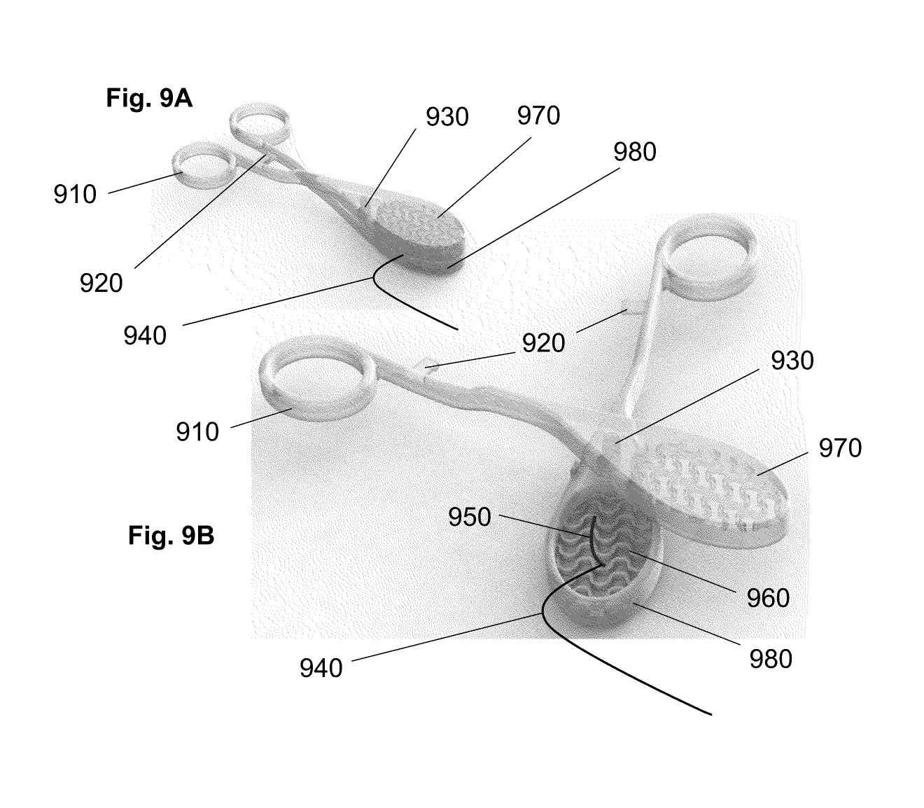

FIGS. 9A and 9B illustrate a prototype capture device that includes scissor-type locking forceps handles, according to one embodiment of the invention.

FIG. 9A shows the “closed” position. The scissor-style handles 910 are held firmly in the closed position by a locking mechanism 920 common to scissor-type forceps. A pivot-type hinge 930 connects two very similar halves 970 and 980 of the device and allows the halves to move with respect to each other when not a closed or locked position. A segment of suture material 940 is seen extending out of the device, where one end of the suture material remains attached to a suture needle captured inside the device and the other end of the suture material remains attached to a surgical site or wound of a patient (not shown).

FIG. 9B shows the “open” position. In this position, the scissor-style handles 910 are far apart and each includes one part of a locking mechanism 920 common to scissor-type forceps. A pivot-type hinge 930 connects two very similar halves of the device and allows the halves to move with respect to each other. A segment of suture material 940 is seen extending out of the device, where one end of the suture material remains attached to a suture needle 950 anchored or embedded in resilient ribs 960 inside the device, and the other end of the suture material remains attached to a surgical site or wound of a patient (not shown).

The prototype illustrated in FIG. 9 appears relatively thick but may be 2-20 mm, 4-18 mm, 5-16 mm, 6-14 mm, or 8-10 mm thick when closed, for example.

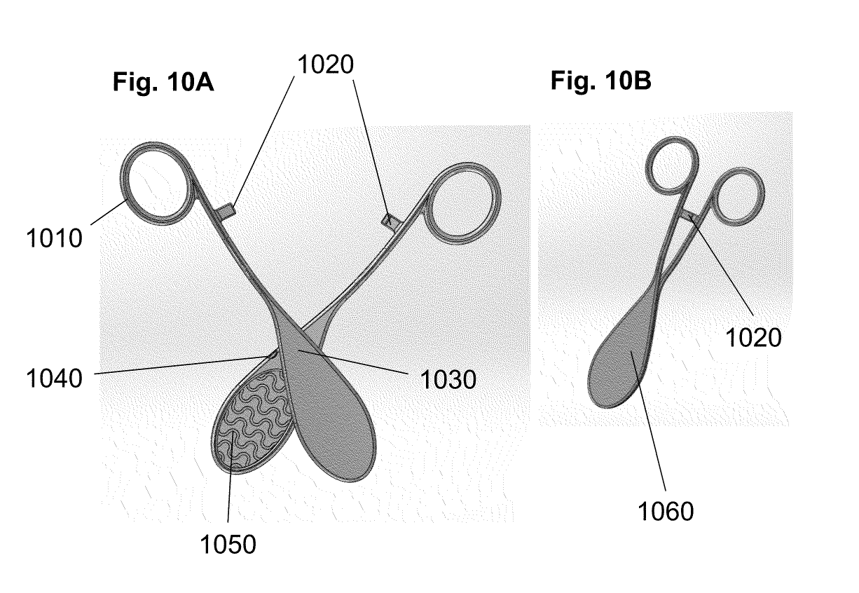

FIGS. 10A and 10B illustrate a prototype capture device that is slightly different from the one illustrated in FIGS. 9A and 9B but also includes scissor-type locking forceps handles, according to another embodiment of the invention. This prototype is flatter than the prototype illustrated in FIGS. 9A and 9B. For example, this prototype may be 2-8 mm, 3-7 mm, or 4-6 mm thick when closed.

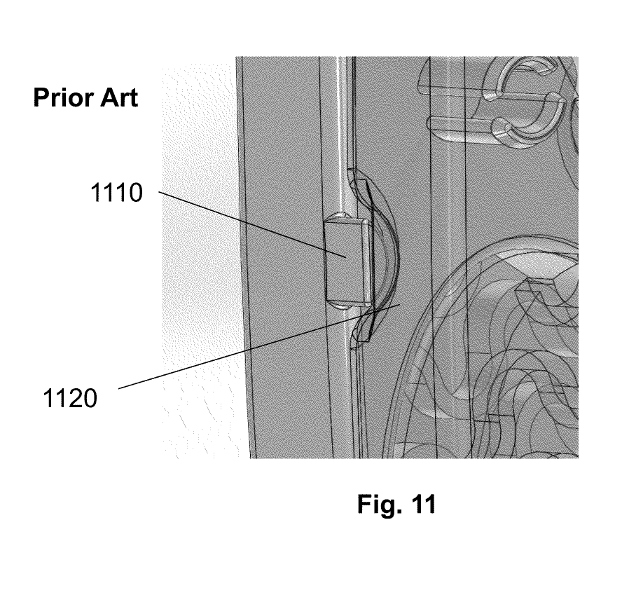

FIG. 10A shows the open configuration and highlights the closing features designed into the forceps. At the edge of one half of the container portion is an end stop 1040 that is designed to prevent the forceps from being pushed past the closed position. A close up of this feature is illustrated in FIG. 11. A locking device common to scissor-type forceps 1020 is also included. The hinge 1030 that connects the two halves is not visible from the outside but is placed in a position corresponding to that shown in FIGS. 9A and 9B. The textured surfaces 1050 or ribs on the inside of the container portion of the device are also shown in the open configuration.

FIG. 10B shows the closed configuration, wherein the common locking device 1020 is in the locked position. This configuration also shows the very flat profile 1060 of this prototype as compared to the thicker prototype shown in FIGS. 9A and 9B. Such a flattened profile may be more easily wrapped into bandages.

FIG. 11 illustrates a close up showing detail of an “end stop” 1110 feature of the prototype in FIG. 10 (labeled in FIG. 10 as 1040), according to embodiments of the invention. In this end stop close up, the forceps are in the closed configuration and the upper forcep 1120 is transparent, so only the outlines can be seen. This demonstrates how the endstop components fit together.

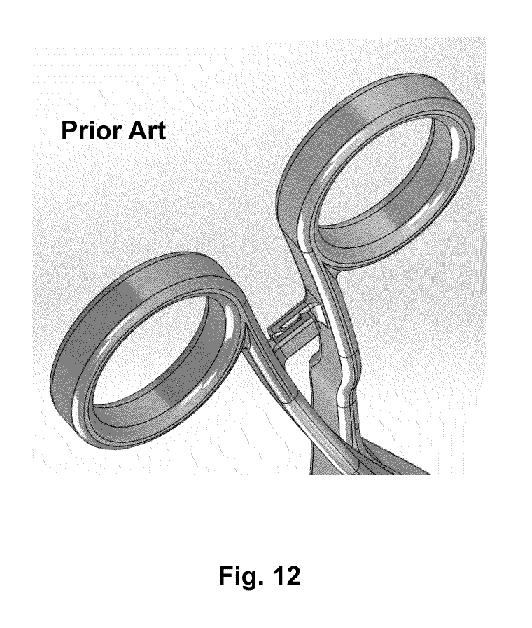

FIG. 12 illustrates a close up showing detail of the common locking mechanism for scissor-type forceps handles featured in the prototypes shown in FIG. 9 and FIG. 10, according to embodiments of the invention.

Methods of the present invention may be performed using any devices of the present invention. Similarly, a system that utilizes the present invention may advantageously include any of the devices of the present invention.

Having described the many embodiments of the present invention in detail, it will be apparent that modifications and variations are possible without departing from the scope of the present invention defined in the appended claims. Furthermore, it should be appreciated that all examples in the present disclosure, while illustrating many embodiments of the present invention, are provided as non-limiting examples and are, therefore, not to be taken as limiting the various aspects so illustrated.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof. References cited herein are also incorporated by reference as though fully set forth herein. The present invention may be better understood by referring to the following non-limiting examples.

### EXAMPLES

### Example 1

### Disposable Clamshell Capture Device

The clamshell type capture device illustrated in FIGS. 1 and 2 contains an array of thin plastic ribs to hold a needle in place. The device captures a suture needle completely by enclosing and stabilizing the needle with support ribs. The device has two tabs on the sides so that it can be picked up using a hemostat and held while the needle is placed. The design is symmetrical for use by right- or left-handed users. Placing the needle in the capture device requires the user to pierce the needle through any location along the array of ribs. Once the tip is secured, the needle driver can be released, and the clamshell capture device can be pinched shut to secure the needle position.

The capture device has a living hinge for opening and closing the device. The case may be secured with a releasable snap latch. This latch may be reopened when access to the needle is needed. Additional ribs located inside the clamshell effectively raise the needle from the inside surface. This facilitates grasping the needle with a needle driver when the needle is to be removed from the capture device. When the needle is stored inside, the suture material extends from the device to the surgical/trauma site and a part of the suture material is secured within the capture device. The device enclosing the needle, with suture material extending to the unfinished procedure site, is conveniently stored in bandages nearby.

This capture device is made from soft plastic such as polyetheylene or polypropylene. It may be mass-produced for disposable single use. The capture device may be a range of different sizes to accommodate needles of different shapes and sizes.

### Example 2

### Flat Rectangular Capture Device

The capture device illustrated in FIG. 3 may be disposable or reusable, depending on the materials from which it is made. This device features over-molded rubber bumpers on the inside surfaces for firmly securing a suture needle between the surfaces. When the needle is stored inside, the suture material extends from the device to the surgical/trauma site and part of the suture material is secured within the capture device.

### Example 3

### Duck Bill Capture Device

The capture device illustrated in FIGS. 4 and 5 is shaped somewhat like a duck bill. The operational ends must be pinched together to open the device such that a suture needle may be inserted. Once the needle is in place, the duck bill ‘jaws’ are closed by releasing the operational ends. Again, when the needle is inside, the suture material extends out to the wound.

### Example 4

### Mid-Needle Exposed Capture Device

The capture device illustrated in FIGS. 6 and 7 is designed to hold the ends of a curved suture needle while leaving the middle of the needle exposed. This allows a needle driver to grasp the middle part of the needle before, during, and after storage of the needle in the device. Again, when the needle is captured, the suture material extends to the wound.

### Example 5

### Other Designs for Capture Devices

The capture device illustrated in FIG. 8 is a twist-top cylinder. Many other designs could also function as suture needle capture devices, including but not limited to containers similar to cosmetic compacts, screw-cap or snap-cap bottles, jars, or plastic cylinders, a matchbox, stacking plates with LEGO®-style interlocking posts for capturing a suture needle between plates (the plates and the posts could be any geometric configuration), or a change purse style container that is pinched at the sides to open.

### Example 6

### Device with Scissor-Style Handles

The capture devices illustrated in FIGS. 9-12 include scissor-type locking forceps handles which are common to many medical instruments. The handles are attached by a pivot-type hinge to the two halves of the container part of the capture device. Some versions of this prototype are thicker at the container part and may be partially or completely made of disposable materials. Other versions may include stainless steel or other harder materials (e.g., hard plastics) and therefore can be thinner without increased risk of having a needle penetrate the container part. Capture devices made of some harder materials such as hard plastics may also be disposable.

As with the other capture device designs, ribs made of resilient materials inside the container part of the device allow the user to pierce and anchor the needle into the interior of the device container. The suture material extends out of the device and remains attached to the wound or surgical site on the patient, and the device may be conveniently stored in bandages near the site. When it is time for suturing to be completed, the user locates and opens the device, retrieves the suture needle (e.g., with a needle driver), and resumes stitching the incision or wound.

1. A device for storing a needle that is fixedly attached at one end to a suture material comprising: a container for enclosing the needle and a first portion of the suture material, wherein the container comprises:
(i) a component for securing the needle inside the container; and
(ii) a mechanism for opening and closing the container;
wherein the container is configured to allow a second portion of the suture material to extend from the container when the container is in the closed position and contains a needle.

2. The device of claim 1, wherein the needle is a surgical suture needle.

3. The device of claim 1, wherein the needle is a swaged needle.

4. The device of claim 1, further comprising a component inside the container that is configured to be pierced by the end of the needle.

5. The device of claim 1, wherein the component for securing the needle and the first portion of the suture material inside the container is configured to contain the needle between two elements that secure the needle in a fixed position.

6. The method of claim 5, wherein the component configured to contain the needle between the two securing elements comprises at least two protrusions configured to contain the needle between the protrusions.

7. The device of claim 1, further comprising a component for securing the first portion of the suture material in a fixed position in the container.

8. The device of claim 1, wherein the mechanism for opening and closing the container comprises a component for securing the container in a closed position.

9. The device of claim 1, wherein the container is configured to comprise a top portion and a bottom portion connected to the mechanism for opening and closing the container.

10. The device of claim 9, wherein the top portion and the bottom portion are configured as mirror images of each other.

11. The device of claim 9, wherein the mechanism for opening and closing the container comprises a hinge.

12. The device of claim 1, wherein the mechanism for opening and closing the container comprises at least one handle.

13. The device of claim 12, wherein a first handle is attached to a first portion of the container and the second handle is attached to a second portion of the container.

14. The device of claim 9, wherein the first portion of the device and the second portion of the device are connected by a cylindrical component configured to allow inward facing surfaces of the first and second portions of the container to be positioned adjacent to one another so as to close the container.

15. The device of claim 1, wherein at least part of the container is sterile.

16. The device of claim 1, further comprising a latch or lock.

17. The device of claim 1, wherein pressure must be applied to the device to open it.

18. The device of claim 1, wherein pressure must be applied to the device to close it.

19. The device of claim 1, further comprising a notch where the middle part of the needle is exposed when the needle is captured in the closed device.

20. A method for temporarily storing a suture needle attached to suture material within a device according to claim 1, comprising inserting a suture needle and some portion of attached suture material into the capture device and closing the capture device.

21. The method of claim 20, wherein at least the sharp point of the needle is secured within the device.

22. The method of claim 21, wherein the needle is fully enclosed within the device.
